(19) United States
(12) Patent Application Publication
(10) Pub. No.: US 2025/0035602 A1
(43) Pub. Date: Jan. 30, 2025

(54) METHOD FOR AUTOMATICALLY IDENTIFYING EMISSION SOURCES IN SOURCE APPORTIONMENT PROCESS OF POLLUTANTS

(71) Applicant: Nankai University, Tianjin (CN)

(72) Inventors: Guoliang SHI, Tianjin (CN); Bo XU, Tianjin (CN); Zhenyu WANG, Tianjin (CN); Yinchang FENG, Tianjin (CN)

(21) Appl. No.: 18/487,185

(22) Filed: Oct. 16, 2023

(30) Foreign Application Priority Data
Jul. 25, 2023 (CN) 202310916095.0

(51) Int. Cl. G01N 33/00; G16C 20/70

(52) U.S. Cl. CPC G01N 33/0062; G16C 20/70

(57) ABSTRACT

A method for automatically identifying emission sources in a source apportionment process of pollutants is provided, which relates to the field of air pollution prevention and control. The method includes: integrating measured source spectrum data and factor spectrum data to generate a labeled data set and an unlabeled data set, respectively; preprocessing the labeled data set to generate a continuous labeled data set; constructing a tree classification model based on the continuous labeled data set; optimizing the tree classification model to determine the optimized tree classification model; coupling the optimized tree classification model and a pseudo-labeling algorithm to generate an integrated model based on the unlabeled data set to automatically identify factor profiles in the unlabeled data set; and determining types of the emission sources based on the factor profiles. The factor profiles can be automatically identified, so that types of emission sources can be quickly determined.

### CROSS-REFERENCE TO RELATED APPLICATION

This patent application claims the benefit and priority of Chinese Patent Application No. 2023109160950 filed with the China National Intellectual Property Administration on Jul. 25, 2023, the disclosure of which is incorporated by reference herein in its entirety as part of the present application.

### TECHNICAL FIELD

The present disclosure relates to the field of air pollution prevention and control, in particular to a method for automatically identifying emission sources in a source apportionment process of pollutants.

### BACKGROUND

The present disclosure relates to the field of air pollution prevention and control, in particular to a method for automatically identifying emission sources in a source apportionment process of pollutants.

### BACKGROUND

As a main atmospheric environmental problem, pollution of fine particles (PM_{2.5}, particles with an aerodynamic diameter less than 2.5 microns) can result in many adverse effects, such as degrading atmospheric visibility, reducing air quality, modulating climate, destroying ecosystems, and endangering human health. In order to mitigate PM_{2.5} pollution and improve air quality, a series of extremely strict clean air action plans have been implemented, which have significantly reduced the mass concentration of PM_{2.5}. Identifying the source of pollutants in advance plays an important role in controlling air pollution. A source apportionment method is an effective tool for quantitative apportionment of pollution sources through chemical, physical, mathematical, and other methods. The results of source apportionment (including the types and contributions of emission sources) can provide an important reference for decision-making. Giving priority to governing emission sources which result in serious pollution can effectively control air pollution and improve air quality.

In recent decades, receptor models have been widely used in the source apportionment of pollutants. Experts have also developed a variety of receptor models to solve different environmental problems, such as a positive matrix factorization model (PMF). The receptor model can decompose the chemical composition data of PM_{2.5} into a factor profile matrix and a factor contribution matrix by matrix decomposition. Identifying factors in the factor profiles as emission sources is an important step in the process of source apportionment. The identified factor profiles can be further optimized and stretched. However, the identification process of factor profiles is usually highly dependent on artificial experience or prior knowledge, which limits the popularization and application of the receptor model to some extent. Generally speaking, rich experience comes from experts' in-depth understanding for the characteristics of emission sources. The process of artificial identification is subjective and requires extensive knowledge. Users of the model will spend a lot of time to adjust the parameters. The standards of factor identification also vary, and different people may obtain different results, resulting in deviations of the results. In addition, artificial identification is not conducive to obtaining real-time source apportionment results in the haze episodes, especially for online datasets. Therefore, it is an important and urgent problem to identify factors in the factor profiles quickly and correctly.

### SUMMARY

The objective of some embodiments of the present disclosure is to provide a method for automatically identifying emission sources in a source apportionment process of pollutants, so as to solve the problem of deviations in the identification results of the artificially identified factor profiles.

In order to achieve the above objective, the present disclosure provides the following technical solution.

The present disclosure provides a method for automatically identifying emission sources in a source apportionment process of pollutant, comprising:
integrating measured source profiles and factor profiles to generate a labeled data set and an unlabeled data set, respectively: where the measured source profiles are priori knowledge, which are derived from actually measured samples of the emission sources and are configured for revealing physical and chemical features of the emission sources;
preprocessing the labeled data set to generate a continuous labeled data set;
constructing a tree classification model based on the continuous labeled data set;
optimizing the tree classification model to determine the optimized tree classification model;
coupling the optimized tree classification model and a pseudo-labeling algorithm to generate an integrated model based on the unlabeled data set, so as to automatically identify factor profiles in the unlabeled data set; and
determining types of the emission sources according to the factor profiles.

Preferably, preprocessing the labeled data set to generate the continuous labeled data set specifically includes:
oversampling the measured source profiles in the labeled data set to generate oversampled measured source profiles;
normalizing independent variables of the oversampled measured source profiles to generate normalized measured source profiles; and
encoding dependent variables of the normalized measured source profiles to form a continuous labeled data set.

Preferably, constructing the tree classification model based on the continuous labeled data set specifically includes:
dividing the continuous labeled data set into a training data set and a testing data set;
training a plurality of the machine learning models by using the training data set to generate a plurality of the trained machine learning models;
testing each of the trained machine learning models by using the testing data set to generate evaluation indexes, where the evaluation indexes comprise accuracy, a precision rate and a recall rate; and
screening one of the machine learning models as the tree classification model based on all of the evaluation indexes.

Preferably, optimizing the tree classification model to determine the optimized tree classification model specifically includes:
traversing a gradient change of key parameters of the optimized tree classification model to determine optimal key parameters, where the key parameters comprise the number of decision trees and the maximum number of features; and
optimizing the tree classification model based on the optimal key parameters to determine the optimized tree classification model.

Preferably, coupling the optimized tree classification model and the pseudo-labeling algorithm to generate the integrated model based on the unlabeled data set to automatically identify the factor profiles in the unlabeled data set specifically includes:
screening factor profiles with prediction probabilities greater than a predetermined probability from the unlabeled data set by using the integrated model;
assigning pseudo labels to the screened factor profiles by using the pseudo-labeling algorithm;
adding a data set of the factor profiles assigned with the pseudo labels to the training data set to form a new training data set;
constructing a new tree classification model based on the new training data set to identify remaining factor profiles in the unlabeled data set.

According to the specific embodiments provided by the present disclosure, the present disclosure discloses the following technical effects. The present disclosure provides a method for automatically identifying emission sources in a source apportionment process of pollutants. The method constructs and optimizes a tree classification model based on the prior knowledge, and couples the optimized tree classification model with the pseudo-labeling algorithm to generate an integrated model, so as to automatically identify the factor profiles in the non-labeled data set, quickly identify types of emission sources, and provide guidance for the identification of pollutant sources in a complex environment.

### BRIEF DESCRIPTION OF THE DRAWINGS

In order to explain the technical solutions in embodiments of the present disclosure or in the prior art more clearly, the accompanying drawings required in the embodiments will be briefly introduced hereinafter. Apparently, the accompanying drawings in the following description show only some embodiments of the present disclosure. For those skilled in the art, other drawings can be derived from these drawings without creative labor.

FIG. 1 is a flowchart of a method for automatically identifying emission sources in a source apportionment process of pollutant according to Embodiment 1 of the present disclosure.

FIG. 2 is a flowchart of a method for automatically identifying emission sources in a source apportionment process of pollutant according to Embodiment 2 of the present disclosure.

FIG. 3 is a diagram of measured source profiles.

FIG. 4 is a comparison diagram of accuracy of various classification models.

FIG. 5 is a comparison diagram of precision of various classification models.

FIG. 6 is a comparison diagram of recall rates of various classification models.

FIG. 7 is a comparison diagram between a tree classification model and an optimized tree classification model.

FIG. 8 is a diagram of factor profiles identification results.

FIG. 9 is a comparison diagram between factor profiles results identified by the present disclosure and factor profiles results artificially identified.

### DETAILED DESCRIPTION OF THE EMBODIMENTS

The technical solutions in the embodiments of the present disclosure will be clearly and completely described with reference to the drawings in the embodiments of the present disclosure hereinafter. Apparently, the described embodiments are only some embodiments of the present disclosure, rather than all of the embodiments. Based on the embodiment of the present disclosure, all other embodiments obtained by those skilled in the art without creative labor fall within the scope of protection of the present disclosure.

The objective of embodiments of the present disclosure is to provide a method for automatically identifying emission sources in a source apportionment process of pollutants, which can automatically identify factor profiles to quickly determine the types of emission sources.

In order to make the above objective, features and advantages of the present disclosure clearer and more comprehensible, the present disclosure will be explained in further detail with reference to the drawings and specific embodiments hereinafter.

### Embodiment I

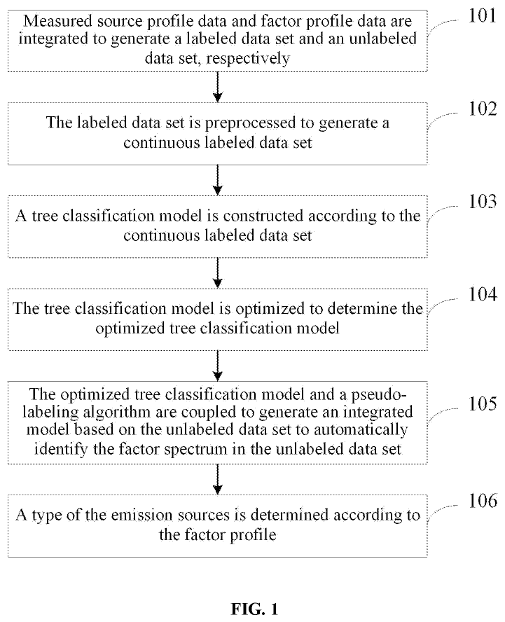

As shown in FIG. 1, the present disclosure provides a method for automatically identifying emission sources in a source apportionment process of pollutants, which includes the following steps 101-106.

In step 101, measured source profiles and factor profiles are integrated to generate a labeled data set and an unlabeled data set, respectively: the measured source profiles are priori knowledge, which are derived from actually measured samples of the emission source and are configured for revealing physical and chemical features of the emission sources.

In practical application, samples and chemical components of the measured source profiles are screened. The measured source profiles can be used as input data of the tree classification model, so that the tree classification model mines the features of the measured source profiles. The factor profile is integrated as prediction data to be identified by the tree classification model.

In step 102, the labeled data set is preprocessed to generate a continuous labeled data set.

In practical application, the step 102 specifically includes: oversampling the measured source profiles in the labeled data set to generate oversampled measured source spectrum data; normalizing an independent variable of the oversampled measured source spectrum data to generate normalized measured source profiles; and encoding a dependent variable of the normalized measured source profiles to form a continuous labeled data set.

In practical application, the preprocessing of the measured source profiles includes oversampling of the samples, normalizing of the independent variables, and label encoding of the dependent variables.

Firstly, the oversampling algorithm is used to synthesize some new samples of a minority class to supplement the number of samples of the minority class and realize sample balance:

$${x}_{n e w} = {x}_{i} + r a n d ( 0 , 1 ) \times ( {x}_{N N} - {x}_{i} ) \qquad ( 1 )$$

where x_{new} represents a newly generated sample: xᵢ represents a sample of the minority class: x_{NN} represents a nearest neighbor sample of xᵢ.

Then, the independent variables of different dimensions are uniformly transformed into the same dimension by using a z-score normalization method to ensure the comparability between independent variables:

$${x}_{j} = \frac{{x}_{j} - {u}_{j}}{{σ}_{j}} \qquad ( 2 )$$

where xⱼ represents each sample: uⱼ represents an average value of all samples j; σⱼ represents a standard deviation of all samples j.

Finally, the dependent variables are encoded by a label encoder algorithm to form a continuous data set, which is convenient for the construction of the machine learning model.

In step 103: a tree classification model is constructed according to the continuous labeled data set.

In practical application, the step 103 specifically includes: dividing the continuous labeled data set into a training data set and a testing data set: training a plurality of the machine learning models by using the training data set to generate a plurality of trained machine learning models: testing each of the trained machine learning models by using the testing data set to generate evaluation indexes, where the evaluation indexes include accuracy, a precision rate and a recall rate; and screening one of the machine learning models as the tree classification model according to all evaluation indexes.

In practical application, the continuous labeled data set is divided into a training data set and a testing data set by using a 10-fold cross validation method. 90% of the samples are selected as the training data set to fit the model, and the remaining 10% are selected as the testing data set to evaluate the performance of the model.

A plurality of the machine learning models are trained by the training data set. Then, based on the trained models, the evaluation indexes (the accuracy, the precision rate and the recall rate) of the models are calculated by using the testing data set of the measured source profiles. The appropriate machine learning model is obtained by comparing the evaluation indexes, and finally the tree classification model is selected.

In step 104, the tree classification model is optimized to determine the optimized tree classification model.

In practical application, the parameters of the machine learning model play an important role in improving the performance of the model. The key parameters of the model include the number of decision trees and the maximum number of features. In the present disclosure, the optimal parameters are obtained by traversing a gradient change of the key parameters of the optimized tree classification model, so as to optimize the model.

The step 104 specifically includes: traversing the gradient change of the key parameters of the optimized tree classification model to determine the optimal key parameters, where the key parameters include the number of decision trees and the maximum number of features; and optimizing the tree classification model according to the optimal key parameters to determine the optimized tree classification model.

In Step 105, the optimized tree classification model and a pseudo-labeling algorithm are coupled to generate an integrated model based on the unlabeled data set, so as to automatically identify factor profiles in the unlabeled data set.

In practical application, the step 105 specifically includes: screening factor profiles with prediction probabilities greater than a predetermined probability from the unlabeled data set by using the integrated model: assigning pseudo labels to a data set of the screened factor profiles by using the pseudo-labeling algorithm: adding the data set of the factor profiles assigned with the pseudo labels to the training data set to form a new training data set; and constructing a new tree classification model according to the new training data set to identify the remaining factor profiles in the unlabeled data set.

In practical application, the tree classification model is coupled with the pseudo-labeling algorithm to form an integrated model. The integrated model is trained by the training data set, and the factor profiles are identified.

Furthermore, samples with a probability value greater than 0.6 (high prediction confidence) are screened out by the integrated model. It is assumed that the prediction results of these samples are correct, and pseudo label are assigned to the screened samples. Then, the screened samples are added to the training data set to form a new training data set, and the remaining factor profiles are re-identified until the factor profiles are identified or the model performance remains unchanged.

In step 106, a type of the emission source is determined according to the factor profiles.

In the present disclosure, the tree classification model coupled with the pseudo-labeling algorithm can automatically, quickly and accurately identify factors in the factor profiles, avoid subjectivity of artificial identification, improve stability and physical significance of identification results of the factor profiles, save a lot of time for users of the source apportionment model, and also lay a solid foundation for the subsequent stretching of the factor profiles and the calculation of the source apportionment results.

In the present disclosure, the input data is preprocessed in various ways, so that the model can obtain data information more quickly. The oversampling algorithm solves the imbalance problem of different types of data. The z-score normalization method transforms independent variables of different dimensions into independent variables of the same dimension uniformly, so as to ensure the comparability between independent variables. The label encoder algorithm encodes dependent variables to form a continuous data set, which is convenient for the construction of the machine learning model.

In the present disclosure, the optimized key parameters of the tree classification model are traversed, so that the tree classification model can be continuously and iteratively optimized, the prediction accuracy of the tree classification model can be optimized, and the performance of the tree classification model can be improved.

### Embodiment II

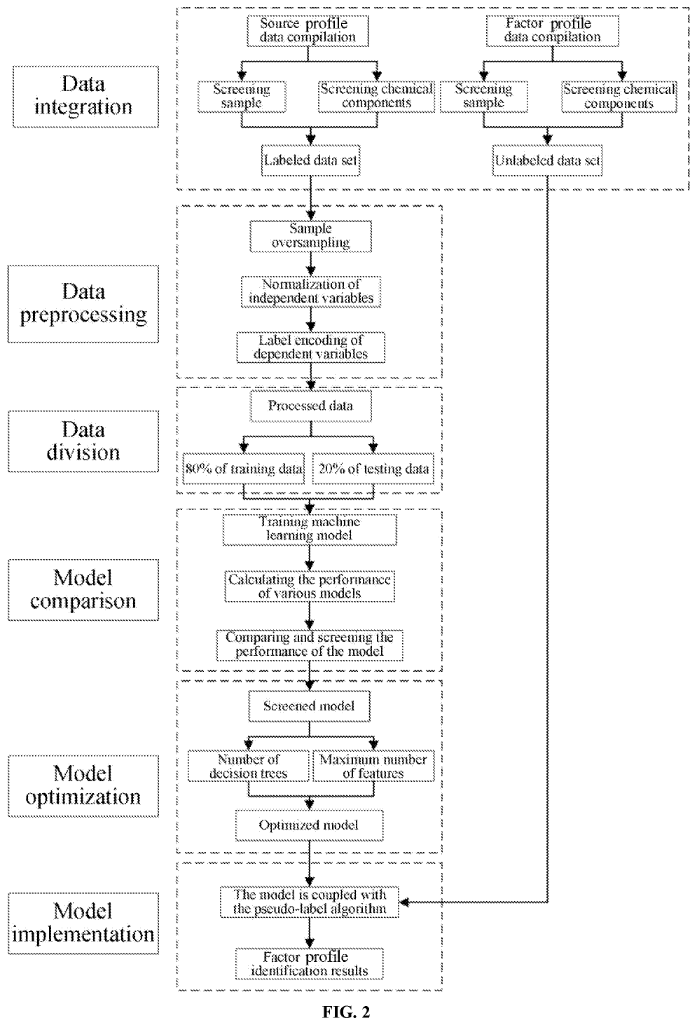

With Tianjin as a research area, the method according to Embodiment 1 is implemented, as shown in FIG. 2. The specific steps are as follows.

1. The measured source profiles and the factor profiles are integrated.

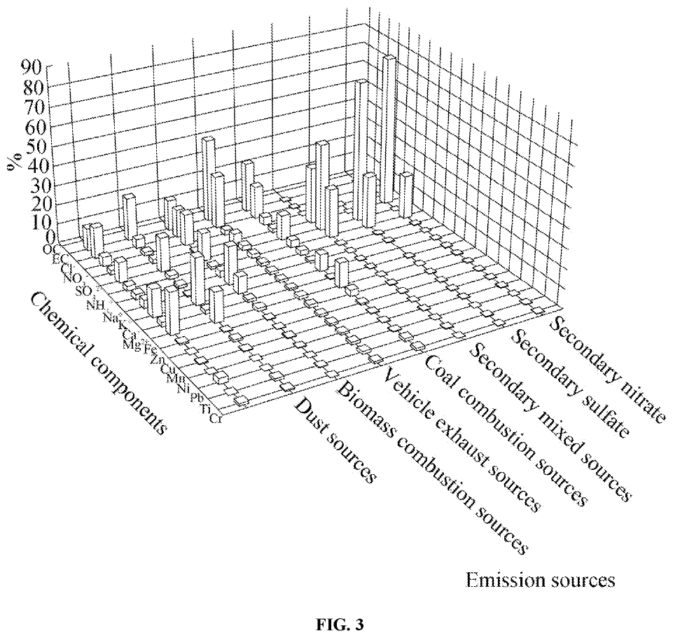

The measured source profiles reflect the features of the emission sources and can be used as the basis for identifying the factor profiles. The percentages of chemical components in the measured source profiles are shown in FIG. 3.

A total of 246 samples are screened. Each emission source contains 18 chemical components (inclusive). The labeled components of each emission source are different. OC (25.5%), EC (17.0%), SO₄²⁻ (13.2%), Fe (12.8%) and Ca²⁺ (8.9%) are the main chemical components, which account for a relatively high proportion in coal combustion sources. In vehicle exhaust sources, carbonaceous components account for a relatively high proportion, in which OC and EC are dominant species, accounting for 42.9% and 27.9%, respectively. In biomass combustion sources, K⁺, Cl⁻, OC, EC and SO₄²⁻ account for 20.1%, 16.2%, 15.9%, 14.7% and 14.4%, respectively, which are labeled species. In dust sources, some crustal elements, including Ca²⁺ (24.5%) and Fe (15.6%), OC (22.3%) and SO₄²⁻ (17.6%), are key species. In industrial sources, some elements are important components, such as Fe (21.7%). EC (16.1%), OC (11.6%) and SO₄²⁻ (10.6%) also play an important role. In secondary nitrate, NO₃⁻ and NH₄⁺ are labeled species, accounting for 77.5% and 22.5%, respectively. In secondary sulfates, SO₄²⁻ and NH₄⁺ are labeled species, accounting for 72.7% and 27.3%, respectively. In secondary mixed sources, SO₄²⁻, NO₃⁻ and NH₄⁺ account for 45.3%, 29.2% and 25.5%, respectively.

2. The measured source profiles are preprocessed.

The preprocessing of measured source profiles can ensure better performance of the machine learning model. In the present disclosure, the data preprocessing method mainly includes oversampling of samples, normalization of independent variables, and label encoding of dependent variables. For the oversampling of samples, considering that the proportion imbalance between categories is a common problem encountered by the machine learning model in dealing with classification problems, unbalanced data affects the prediction of the machine learning model. The large difference in the number of each factor profiles results in the imbalance among samples and affects the performance of the model. The SMOTE algorithm supplements the number of samples of a minority class and balances the number of different sources. Finally, a total of 800 samples are formed as a training data set. For the normalization of independent variables, considering that independent variables have different sizes and there may be significant differences among the values, this may affect the results of data analysis. In order to eliminate the influence of the difference in size and range of values between indexes, it is necessary to normalize the data. The z-score normalization is a commonly used standardization method based on a mean and a variance of data. The processed data conforms to the standard normal distribution, with a mean of 0 and a variance of 1. For the label encoding of dependent variables, it is difficult to deal with the dependent variables mathematically because the dependent variables are text data (characters). The machine learning model is developed based on mathematical principles, and better results can be obtained based on numerical data. Therefore, characters must be mapped to numeric values. The label encoder algorithm can standardize discrete data or characters to encode values between 0) and n−1 (n is the number of classes) to form a continuous data set.

3. The training data is divided.

The training data is divided into a training set and a testing set by using the 10-fold cross validation method. 90% of the samples are selected as the training data set to fit the model, and the remaining 10% of the samples are used as the testing data set to evaluate the performance of the model. There are 720 pieces of data in the training data set and 80 pieces of data in the testing data set.

4. The machine learning model is screened.

There are many available machine learning classification models, including Extra Trees Classifier (ETC). Random Forest Classifier (RFC), XGB Classifier (XGBC), Gradient Boosting Classifier (GBC). K Neighbors Classifier (KNC), and Decision Tree Classifier (DTC).

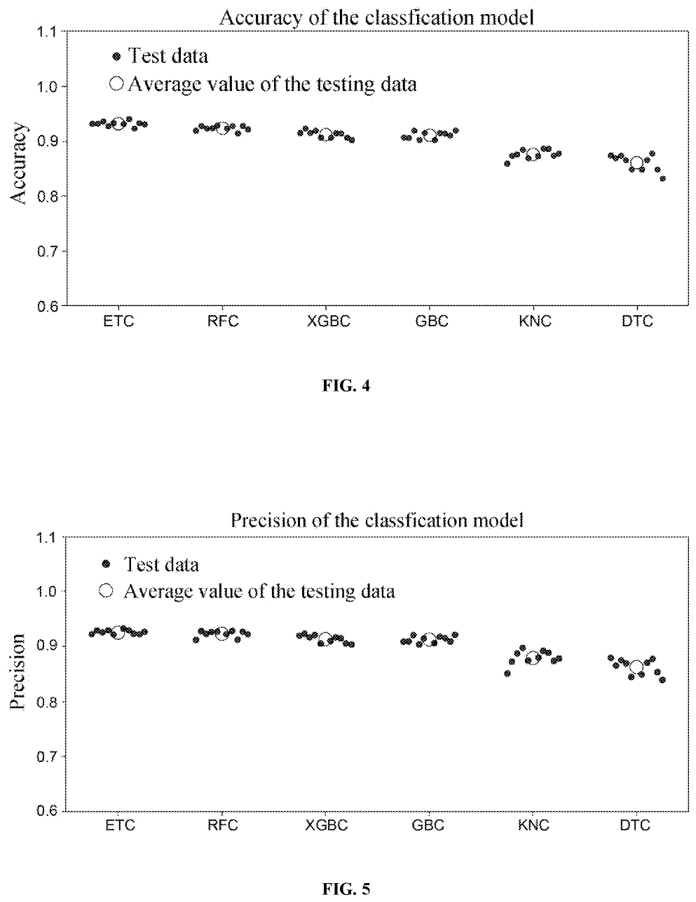

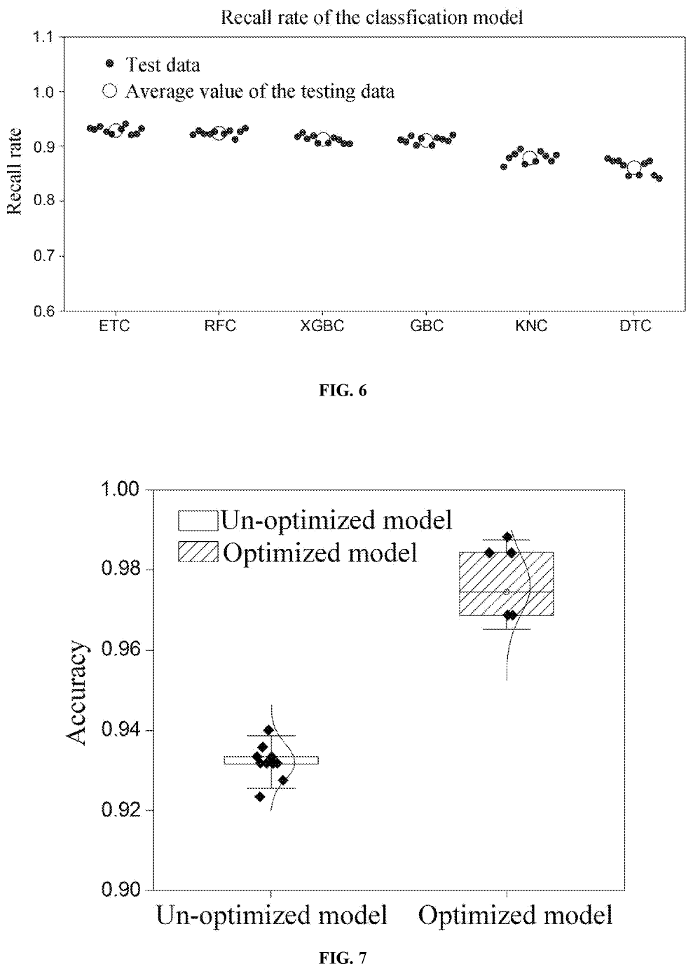

In order to select a suitable model, a plurality of machine learning classification models are compared. The evaluation indexes include (i) accuracy. (ii) precision and (iii) a recall rate, which are selected to evaluate the performance of the model from different perspectives. In order to ensure that the machine learning classification model obtains reliable results, high precision and a high recall rate are necessary. The performance comparison of these classification models is shown in FIG. 4 to FIG. 6.

The results show that ETC has higher accuracy (0.932), higher precision (0.926) and a higher recall rate (0.930). The performance of ETC is better than other models, and its reliability is higher. Therefore, the ETC model is finally selected to construct this machine learning model.

5. The machine learning model is optimized.

The performance of the model depends on parameters (mainly including the number of decision trees and the number of features). The optimal parameters can be obtained by random search with the 10-fold cross validation method. When the number of decision trees is 15, the model has the optimal performance. The number of features has little influence on the performance of the model, and the number of features is finally determined to be 4. The comparison between the un-optimized model and the optimized model is shown in FIG. 7. The overall model performance of the optimized model has been improved, with higher accuracy (0.988), higher precision (0.987) and a higher recall rate (0.988).

6. The optimized machine learning model is coupled with the pseudo-labeling algorithm to identify the factor profiles.

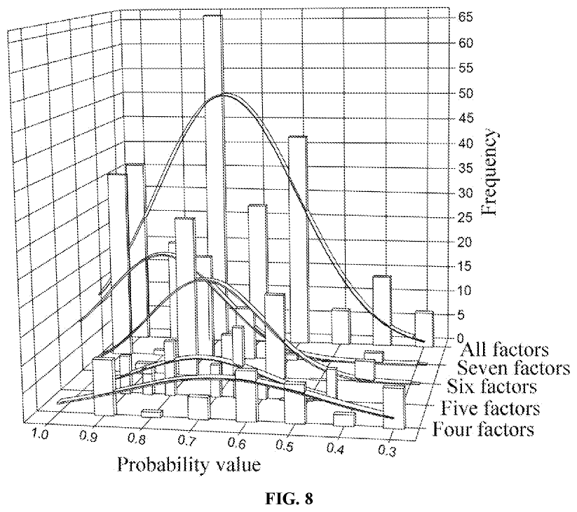

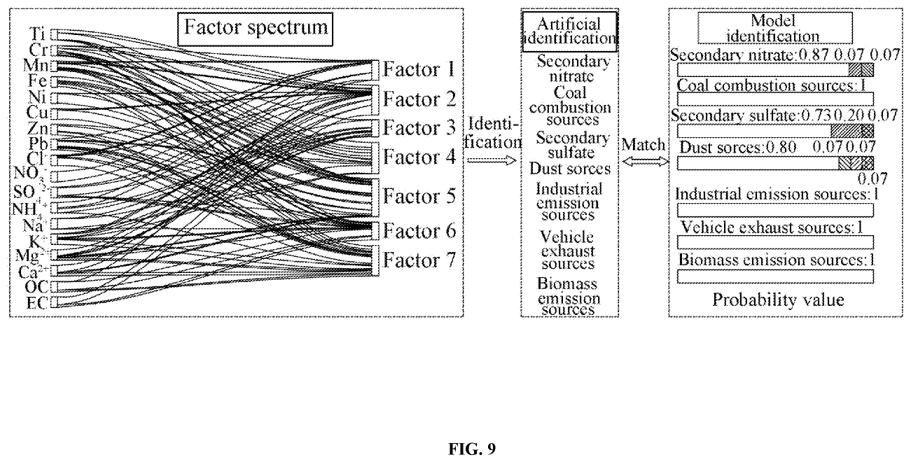

The ETC model coupled with the pseudo-labeling algorithm further identifies the factor profiles, and the integrated model screens out samples with a probability value greater than 0.6 (high prediction confidence). It is assumed that the prediction results of these samples are correct, and pseudo labels are assigned to the screened samples. Then, the screened samples are added to the training data set of the measured source profiles to form a new training data set, and the remaining factor profiles are re-identified until all of the factor profiles are identified or the model performance remains unchanged. The identification results of the integrated model are shown in FIG. 8. The integrated model can identify most of the factor profiles, with an average identification rate of 85.5%. The probability values of the identified factor profiles are mainly concentrated on 0.8. Because the factor profiles are applied to 4 to 7 factors, respectively, the identification rates are 77.5%. 88%. 93.3% and 97.1%, respectively, and the probability values are concentrated on 0.93, 0.67, 0.87 and 1.0, respectively. Therefore, when the number of factors is seven, the identification rate of the model is better, the probability value of the model is higher, and the overall performance of the model is better than other number of factors. In addition, in order to verify the effectiveness of the model, the results of the artificially identified factor profiles are compared with those of the factor profiles identified by the integrated model, and the comparison results are shown in FIG. 9. The comparison results show that the results of model identification has a 100% match with those of artificial identification, which proves the effectiveness of the integrated model.

In this specification, various embodiments are described in a progressive way. Each embodiment focuses on the difference from other embodiments, and the same and similar parts between the embodiments may refer to each other.

In the specification, specific examples are used for illustration of the principles and implementations of the present disclosure. The description of the above embodiments is used to help illustrate the method and core ideas of the present disclosure. In addition, those of ordinary skill in the art can make various modifications in terms of specific implementations and the scope of application in accordance with the ideas of the present disclosure. In conclusion, the contents of the specification should not be construed as limitations to the present disclosure.

What is claimed is:

1. A method for automatically identifying emission sources in a source apportionment process of pollutant, comprising:
integrating measured source profiles and factor profiles to generate a labeled data set and an unlabeled data set, respectively: wherein the measured source profiles are priori knowledge, which are derived from actually measured samples of the emission sources and are configured for revealing physical and chemical features of the emission sources;
preprocessing the labeled data set to generate a continuous labeled data set;
constructing a tree classification model based on the continuous labeled data set;
optimizing the tree classification model to determine the optimized tree classification model;
coupling the optimized tree classification model and a pseudo-labeling algorithm to generate an integrated model based on the unlabeled data set, so as to automatically identify the factor profiles in the unlabeled data set; and
determining types of the emission sources based on the factor profiles.

2. The method according to claim 1, wherein preprocessing the labeled data set to generate the continuous labeled data set comprises:
oversampling a measured source spectrum data in the labeled data set to generate oversampled measured source spectrum data;
normalizing independent variables of the oversampled measured source profiles to generate normalized measured source profiles; and
encoding dependent variables of the normalized measured source profiles to form the continuous labeled data set.

3. The method according to claim 1, wherein constructing the tree classification model based on the continuous labeled data set comprises:
dividing the continuous labeled data set into a training data set and a testing data set;
training a plurality of machine learning models by using the training data set to generate a plurality of trained machine learning models;
testing each of the trained machine learning models by using the testing data set to generate evaluation indexes, wherein the evaluation indexes comprise accuracy, a precision rate and a recall rate; and
screening one of the machine learning models as the tree classification model based on all of the evaluation indexes.

4. The method according to claim 1, wherein optimizing the tree classification model to determine the optimized tree classification model comprises:
traversing a gradient change of key parameters of the optimized tree classification model to determine optimal key parameters, wherein the key parameters comprise a number of decision trees and a maximum number of features; and
optimizing the tree classification model based on the optimal key parameters to determine the optimized tree classification model.

5. The method according to claim 3, wherein coupling the optimized tree classification model and a pseudo-labeling algorithm to generate an integrated model based on the unlabeled data set, so as to automatically identify the factor profiles in the unlabeled data set comprises:
screening factor profiles with prediction probabilities greater than a predetermined probability from the unlabeled data set by using the integrated model;
assigning pseudo labels to the screened factor profiles by using the pseudo-labeling algorithm;
adding a data set of the factor profiles assigned with the pseudo labels to the training data set to form a new training data set;
constructing a new tree classification model based on the new training data set to identify remaining factor profiles in the unlabeled data set.
